(19) United States
(12) Patent Application Publication
(10) Pub. No.: US 2018/0133188 A1
(43) Pub. Date: May 17, 2018

(54) WEIGHT-LOSS BEVERAGE COMPOSITION AND METHOD

(71) Applicant: Jordan McKinnon, Burnaby (CA)

(72) Inventor: Jordan McKinnon, Burnaby (CA)

(21) Appl. No.: 15/350,702

(22) Filed: Nov. 14, 2016

(51) Int. Cl. A61K 31/205; A61K 36/82; A61K 36/74; A61K 31/216; A61K 31/047; A61K 31/196; A61K 36/73; A61K 36/889; A61K 36/38; A61K 36/77; A61K 31/194; A61K 31/19; A61K 9/00; A23L 2/52; A23L 33/175; A23L 33/105; A23L 33/15; A23L 33/00; A23L 33/10

(52) U.S. Cl. CPC A61K 31/205; A23V 2002/00; A61K 36/74; A61K 31/216; A61K 31/047; A61K 31/196; A61K 36/73; A61K 36/889; A61K 36/38; A61K 36/77; A61K 31/194; A61K 31/19; A61K 9/0095; A23L 2/52; A23L 33/175; A23L 33/105; A23L 33/15; A23L 33/30; A23L 33/10; A61K 36/82

(57) ABSTRACT

A weight-loss beverage composition including L-carnitine-L-tartrate, green tea extract, green coffee bean extract/chlorogenic acid, at least one B vitamin, choline bitartrate, inositol, para-aminobenzoic acid, raspberry ketones, acai extract, garcinia cambogia, and guarana. The weight-loss beverage composition is useful for promoting health and weight reduction.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### FIELD OF THE INVENTION

The present invention relates generally to the field of beverages and more specifically relates to a weight-loss beverage.

### DESCRIPTION OF RELATED ART

Inability to properly manage weight is an increasing problem among the adult population as indicated by increasingly rising rates of obesity. Obesity and excessive weight lead to a litany of health problems such as cardiovascular disease, skeletal and joint disease, diabetes, cancer, poor body image, depression and overall decreased energy. However, maintaining proper body weight is difficult for individuals who lack the time to exercise or the knowledge and wherewithal to abide by a healthy diet.

Weight gain is a complex problem. The health-conscious public is concerned about excessive weight gain, but does not generally want to maintain strict diets to lose or maintain a healthy weight. Individuals find problems ingesting the appropriate nutrients and vitamins in order to improve their weight and health. Therefore, a composition is desired to provide the needed ingredients at the right quantities to promote weight loss and health that so many diets presently are lacking.

Several attempts have been made to solve the above-mentioned problems such as those found in U.S. Pat. No. 7,989,007 to Vincent Giuliano relates to a weight loss beverage. The described weight loss composition includes a purposefully formulated weight loss composition and a method for causing a meaningful weight loss in a living human subject. The formulated weight loss composition, in its most preferred embodiments comprises: at least one amino acid that is a pharmacologically active precursor in the catecholamine biosynthesis pathway; one or more sympathomimetic agents; a lipolytic agent; a high-Dalton macromolecular protein, and a plurality of micronutrients. The preferred composition is formulated as a dry particulate admixture initially; and then is made into a fluid beverage, which is able to achieve multiple physiologic functions in-vivo. These in-vivo functions include: causing a meaningful weight loss, with a preference for lipolysis and fat metabolism; providing for a low glycemic index; offering lean muscle maintenance and replenishment; and concomitantly initiating dopaminergic (D2-receptor) brain stimulation, resulting in improved cognitive function and affective augmentation.

Preferably, a weight-loss beverage should comprise more effective ingredients and be manufactured at a modest expense. Thus, a need exists for weight-loss beverage composition to avoid the above-mentioned problems.

### BRIEF SUMMARY OF THE INVENTION

In view of the foregoing disadvantages inherent in the known a weight-loss beverage art, the present disclosure provides a novel weight-loss beverage composition. The general purpose of the present disclosure, which will be described subsequently in greater detail, is to provide a weight-loss beverage composition.

A weight-loss beverage composition is disclosed herein. The weight-loss beverage composition may comprise L-carnitine-L-tartrate, green tea extract, green coffee bean extract/chlorogenic acid, at least one B vitamin, choline bitartrate, inositol, para-aminobenzoic acid, raspberry ketones, acai extract, garcinia cambogia, and guarana. Furthermore, the weight-loss beverage composition comprises in functional combination the L-carnitine-L-tartrate, the green tea extract, the green coffee bean extract/chlorogenic acid, the at least one B vitamin, the choline bitartrate, the inositol, the para-aminobenzoic acid, the raspberry ketones, the acai extract, the garcinia cambogia, and the guarana thereby creating an admixture.

The L-carnitine-L-tartrate may comprise approximately 100 mg of the admixture. The green tea extract and the green coffee bean extract/chlorogenic acid may separately comprise approximately 80 mg of the admixture. Furthermore, the para-aminobenzoic acid and the guarana may separately comprise approximately 25 mg of the admixture. The raspberry ketones may comprise approximately 20 mg of the admixture. Finally, the garcinia cambogia may comprise approximately 60 mg of the admixture.

Additionally, B1-thiamine and B2-riboflavin may separately comprise approximately 30 mg of the admixture. B3-niacinamide, B5-panthothenic acid, B6-pyridoxine hydrochloride, the choline bitartrate, the inositol, and the acai extract may separately comprise approximately 50 mg of the admixture. B7-biotin, B9-folic acid, and B12-methyl may separately comprise approximately 500 mg of the admixture. The at least one B vitamin may be further understood to include the following B vitamins: B1-thiamine, B2-riboflavin, B3-niacinamide, B5-panthothenic, B6-pyridoxine hydrochloride, B7-biotin, B9-folic acid, and B12-methyl. Moreover, the weight-loss beverage composition includes additional ingredients. Providing an admixture with these ingredients in the specified quantity, may promote human health and weight loss by consumption through the mouth.

According to another embodiment, a method of using a weight-loss beverage composition is also disclosed herein. The method of using the weight-loss beverage composition includes opening bottle comprising the weight-loss beverage composition, consuming the weight-loss beverage composition, and optionally repeating consumption of the weight-loss beverage composition for continuous promotion of health and weight loss.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a weight loss beverage composition, constructed and operative according to the teachings of the present disclosure.

FIG. 1 is a perspective view of the weight-loss beverage composition during an ‘in-use’ condition, according to an embodiment of the disclosure.

FIG. 2 is a front view of the weight-loss beverage composition of FIG. 1, according to an embodiment of the present disclosure.

FIG. 3 is a data table comprising the ingredients of the weight-loss beverage composition of FIGS. 1-2, according to an embodiment of the present disclosure.

FIG. 4 is a data table comprising additional ingredients of the weight-loss beverage composition of FIGS. 1-3, according to an embodiment of the present disclosure.

FIG. 5 is a flow diagram illustrating a method of using the weight-loss beverage composition, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a beverage and more particularly to a weight-loss beverage composition as used to promote the health and weight loss of a user.

Generally, the weight-loss beverage composition may contain lipotropic compounds. Lipotropic compounds help break down the fat during metabolism of the body. For example, choline and inositol are lipotropics that provide a healthy maintenance for the liver. When fat and bile is trapped in the liver, major health concerns may arise. It is essential to maintain a steady intake of nutritional supplements like lipotropic compounds to regulate and promote health and weight reduction.

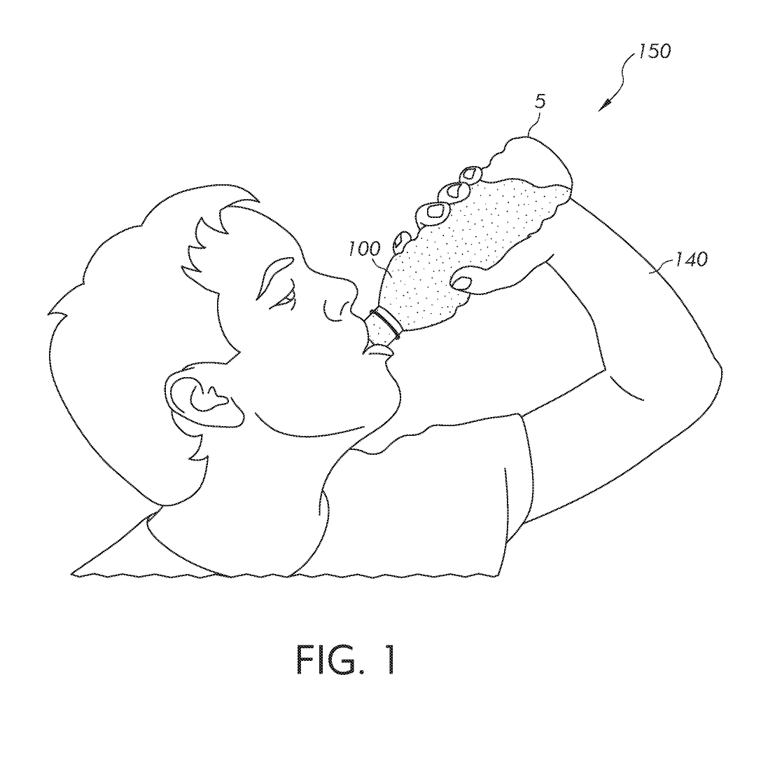

Referring now more specifically to the drawings by numerals of reference, there is shown in FIGS. 1-4, various views of a weight-loss beverage composition 100. FIG. 1 shows the weight-loss beverage composition 100 during an ‘in-use’ condition 150, according to an embodiment of the present disclosure. Here, the weight-loss beverage composition 100 may be beneficial for use by a user 140 to promote health and weight reduction.

As illustrated, the weight-loss beverage composition 100 may include L-carnitine-L-tartrate 102, green tea extract 104, green coffee bean extract/chlorogenic acid 106, at least one B vitamin 108, choline bitartrate 110, inositol 112, para-aminobenzoic acid 114, raspberry ketones 116, acai extract 118, garcinia cambogia 120, and guarana 122. The weight-loss beverage composition 100 comprises in formulaic combination the L-carnitine-L-tartrate 102, the green tea extract 104, the green coffee bean extract/chlorogenic acid 106, the at least one B vitamin 108, the choline bitartrate 110, the inositol 112, the para-aminobenzoic acid 114, the raspberry ketones 116, the acai extract 118, the garcinia cambogia 120, and the guarana 122 thereby creating an admixture. The weight-loss beverage composition 100 is suitably mixed to provide for human consumption for weight reduction.

The L-carnitine-L-tartrate 102 may comprise approximately 100 mg of the admixture. Moreover, the green tea extract 104 and the green coffee bean extract/chlorogenic acid 106 may separately comprise approximately 80 mg of the admixture. The para-aminobenzoic acid 114 and the guarana 122 may separately comprise approximately 25 mg of the admixture. The raspberry ketones 116 may comprise approximately 20 mg of the admixture. In addition, the garcinia cambogia 120 may comprise approximately 60 mg of the admixture.

B1-thiamine and B2-riboflavin may separately comprise approximately 30 mg of the admixture. B3-niacinamide, B5-panthothenic acid, B6-pyridoxine hydrochloride, the choline bitartrate 110, the inositol 112, and the acai extract 118 may separately comprise approximately 50 mg of the admixture. Moreover, B7-biotin, B9-folic acid, and B12-methyl may separately comprise approximately 500 mg of the admixture. Furthermore, the at least one B vitamin 108 may be understood to include the following B vitamins: B1-thiamine, B2-riboflavin, B3-niacinamide, B5-panthothenic, B6-pyridoxine hydrochloride, B7-biotin, B9-folic acid, and B12-methyl.

The weight-loss beverage composition 100 may include additional ingredients 124. The additional ingredients 124 include malic acid/citric acid 126, erythritol 128, stevia 130, natural flavors 132, and natural color 134. The weight-loss beverage composition 100 may promote human health and weight loss by consumption through the mouth.

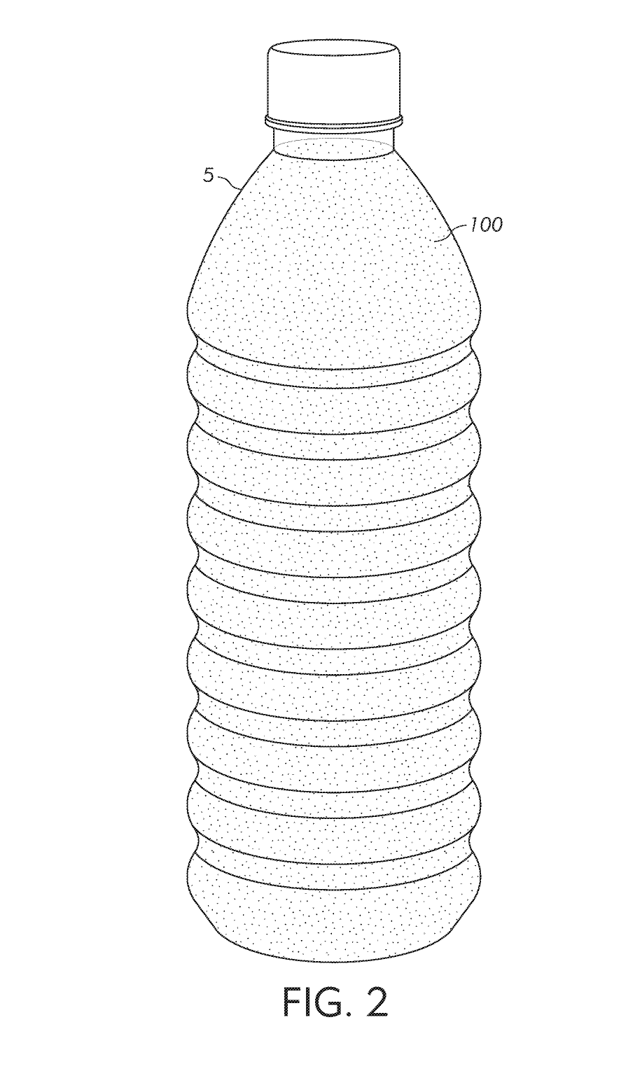

FIG. 2 shows the weight-loss beverage composition of FIG. 1, according to an embodiment of the present disclosure. As above, the weight-loss beverage composition 100 may include the L-carnitine-L-tartrate 102, the green tea extract 104, the green coffee bean extract/chlorogenic acid 106, the at least one B vitamin 108, the choline bitartrate 110, the inositol 112, the para-aminobenzoic acid 114, the raspberry ketones 116, the acai extract 118, the garcinia cambogia 120, and the guarana 122. The weight-loss beverage composition 100 comprises in formulaic combination the L-carnitine-L-tartrate 102, the green tea extract 104, the green coffee bean extract/chlorogenic acid 106, the at least one B vitamin 108, the choline bitartrate 110, the inositol 112, the para-aminobenzoic acid 114, the raspberry ketones 116, the acai extract 118, the garcinia cambogia 120, and the guarana 122 thereby creating an admixture.

Moreover, the weight-loss beverage composition 100 includes additional ingredients 124. The additional ingredients 124 include the malic acid/citric acid 126, the stevia 130, the erythritol 128, the natural flavors 132, and the natural color 134. The weight-loss beverage composition 100 may be mixed together with the proportioned ingredients described in the present embodiments for packaging per bottle 5.

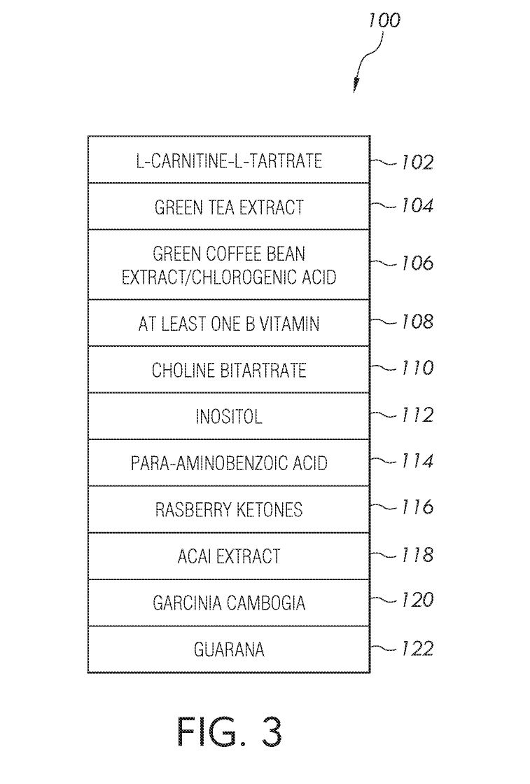

FIG. 3 is a data table comprising the ingredients used in the weight-loss beverage composition 100 of FIG. 1, according to an embodiment of the present disclosure. The weight-loss beverage composition 100 may include the L-carnitine-L-tartrate 102, the green tea extract 104, the green coffee bean extract/chlorogenic acid 106, the at least one B vitamin 108, the choline bitartrate 110, the inositol 112, the para-aminobenzoic acid 114, the raspberry ketones 116, the acai extract 118, the garcinia cambogia 120, and the guarana 122.

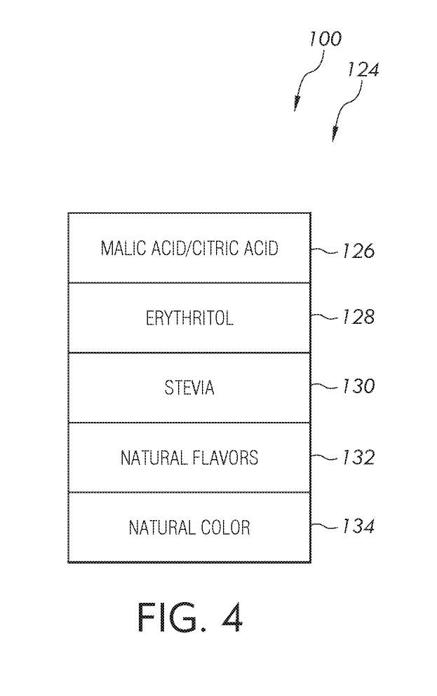

FIG. 4 is a data table comprising the additional ingredients 124 in the weight-loss beverage composition 100 of FIG. 1, according to an embodiment of the present disclosure. The weight-loss beverage composition 100 includes the additional ingredients 124. The additional ingredients 124 may include the malic acid/citric acid 126, the erythritol 128, the stevia 130, the natural flavors 132, and the natural color 134.

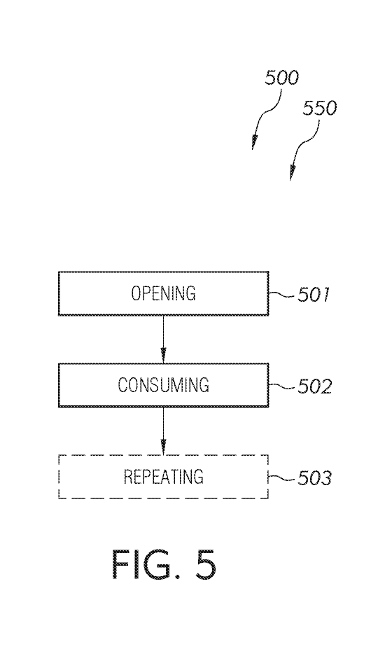

FIG. 5 is a flow diagram illustrating a method for using a weight-loss beverage composition 500, according to an embodiment of the present disclosure. In particular, the method for using a weight-loss beverage composition 500 may include one or more components or features of the weight-loss beverage composition 100 as described above. As illustrated, the method for using a weight-loss beverage composition 500 may include the steps of: step one 501, opening bottle 5 comprising the weight-loss beverage composition 100; step two 502, consuming the weight-loss beverage composition 100; step three 503, repeating consumption of the weight-loss beverage composition 100 for continuous promotion of health and weight loss.

It should be noted that step three 503 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for using the weight-loss beverage composition 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A weight-loss beverage composition having:
L-carnitine-L-tartrate;
green tea extract;
green coffee bean extract/chlorogenic acid;
at least one B vitamin;
choline bitartrate;
inositol;
para-aminobenzoic acid;
raspberry ketones;
acai extract;
garcinia cambogia; and
guarana;
wherein said weight-loss beverage composition comprises in formulaic combination said L-carnitine-L-tartrate, said green tea extract, said green coffee bean extract/chlorogenic acid, said at least one B vitamin, said choline bitartrate, said inositol, said para-aminobenzoic acid, said raspberry ketones, said acai extract, said garcinia cambogia, and said guarana thereby creating an admixture; and
wherein said weight-loss beverage composition is suitably mixed to provide for human consumption for weight reduction.

2. The weight-loss beverage composition of claim 1, wherein said L-carnitine-L-tartrate comprise approximately 100 mg of said admixture.

3. The weight-loss beverage composition of claim 1, wherein said green tea extract and said green coffee bean extract/chlorogenic acid separately comprise approximately 80 mg of said admixture.

4. The weight-loss beverage composition of claim 1, wherein B1-thiamine and B2-riboflavin separately comprise approximately 30 mg of said admixture.

5. The weight-loss beverage composition of claim 1, wherein B3-niacinamide, B5-panthothenic acid, B6-pyridoxine hydrochloride, said choline bitartrate, said inositol, and said acai extract separately comprise approximately 50 mg of said admixture.

6. The weight-loss beverage composition of claim 1, wherein B7-biotin, B9-folic acid, and B12-methyl separately comprise approximately 500 mg of said admixture.

7. The weight-loss beverage composition of claim 1, wherein said para-aminobenzoic acid and said guarana separately comprise approximately 25 mg of said admixture.

8. The weight-loss beverage composition of claim 1, wherein said raspberry ketones comprises approximately 20 mg of said admixture.

9. The weight-loss beverage composition of claim 1, wherein said garcinia cambogia comprises approximately 60 mg of said admixture.

10. The weight-loss beverage composition of claim 1, wherein said weight-loss beverage composition includes additional ingredients.

11. The weight-loss beverage composition of claim 10, wherein the additional ingredients include malic acid/citric acid.

12. The weight-loss beverage composition of claim 10, wherein the additional ingredients include erythritol.

13. The weight-loss beverage composition of claim 10, wherein the additional ingredients include stevia.

14. The weight-loss beverage composition of claim 10, wherein the additional ingredients include natural flavors.

15. The weight-loss beverage composition of claim 10, wherein the additional ingredients include natural color.

16. The weight-loss beverage composition of claim 1, wherein said weight-loss beverage composition promotes human health and weight loss by consumption through the mouth.

17. A weight-loss beverage composition comprising:
L-carnitine-L-tartrate;
green tea extract;
green coffee bean extract/chlorogenic acid;
at least one B vitamin;
choline bitartrate;
inositol;
para-aminobenzoic acid;
raspberry ketones;
acai extract;
garcinia cambogia; and
guarana;
wherein said weight-loss beverage composition comprises in functional combination said L-carnitine-L-tartrate, said green tea extract, said green coffee bean extract/chlorogenic acid, said at least one B vitamin, said choline bitartrate, said inositol, said para-aminobenzoic acid, said raspberry ketones, said acai extract, said garcinia cambogia, and said guarana thereby creating an admixture;
wherein said L-carnitine-L-tartrate comprise approximately 100 mg of said admixture;
wherein said green tea extract and said green coffee bean extract/chlorogenic acid separately comprise approximately 80 mg of said admixture;
wherein B1-thiamine and B2-riboflavin separately comprise approximately 30 mg of said admixture;
wherein B3-niacinamide, B5-panthothenic acid, B6-pyridoxine hydrochloride, said choline bitartrate, said inositol, and said acai extract separately comprise approximately 50 mg of said admixture;
wherein B7-biotin, B9-folic acid, and B12-methyl separately comprise approximately 500 mg of said admixture;
wherein said para-aminobenzoic acid and said guarana separately comprise approximately 25 mg of said admixture;
wherein said raspberry ketones comprises approximately 20 mg of said admixture;
wherein said garcinia cambogia comprises approximately 60 mg of said admixture;
wherein said weight-loss beverage composition includes additional ingredients; and
wherein said weight-loss beverage composition promotes human health and weight loss by consumption through the mouth.

18. The weight-loss beverage composition of claim 17, wherein said additional ingredients includes said malic acid/citric acid, said stevia, said erythritol, said natural flavors, and said natural color.

19. A method of using a weight-loss beverage composition, the method comprising the steps of:
opening a bottle comprising said weight-loss beverage composition; and
consuming said weight-loss beverage composition.

20. The method of claim 19, further comprising the steps of:
repeating consumption of said weight-loss beverage composition for continuous promotion of health and weight loss.
